(19) United States
(12) Patent Application Publication
(10) Pub. No.: US 2014/0277299 A1
(43) Pub. Date: Sep. 18, 2014

(54) APPARATUS AND METHODS FOR TARGETED ULTRAVIOLET PHOTOTHERAPY

(71) Applicant: PHOTOMEDEX, INC., Montgomeryville, PA (US)

(72) Inventors: Alfred J. INTINTOLI, West Chester, PA (US); Phillip SOLOMON, Kibbutz Tzora (IL); Jeffrey I. LEVATTER, Ranch Santa Fe, CA (US); Dolev RAFAELI, Creskill, NJ (US)

(73) Assignee: PHOTOMEDEX, INC., Montgomeryville, PA (US)

(21) Appl. No.: 14/208,060

(22) Filed: Mar. 13, 2014

Related U.S. Application Data
(60) Provisional application No. 61/782,819, filed on Mar. 14, 2013.

(51) Int. Cl. A61N 5/06

(52) U.S. Cl. CPC A61N 5/0616
USPC 607/94

(57) ABSTRACT

An apparatus for applying targeted phototherapy to a skin treatment area includes a UVB module arranged to output light at about 308 nm having at least one flash lamp arranged to generate light including UVB light and a UVB transmissive window for transmitting UVB light generated by the flash lamp, a user interface, and a controller configured to detect when the user interface is actuated and to cause pulsing of the flash lamp in response to the actuation of the user interface. A method of applying targeted phototherapy to a skin treatment area includes contacting a phototherapy apparatus with the skin, the apparatus including at least one flash lamp and a UVB transmissive window, such that the window is positioned in alignment with the treatment area, and activating the flash lamp to cause pulsed UVB light at 308 nm to be transmitted through the window.

### BACKGROUND

The present disclosure relates to phototherapeutic treatment of the epidermis, and more particularly to targeted ultraviolet therapy for treatment of skin conditions. Specifically, methods and apparatus for targeted narrow-band UVB phototherapy are disclosed to adjunctively treat dermatoses (e.g., psoriasis, vitiligo, leukoderma, atopic dermatitis, and alopecia greata).

Approximately 2% of people worldwide have psoriasis, and a lesser but still large percentage have vitiligo and other skin conditions. The condition of psoriasis ranges from mild to severe. It can lead to substantial morbidity and psychological stress and have a profound negative impact on a patient's quality of life. Although available therapies reduce the extent and severity of the disease and improve quality of life, reports have indicated patient dissatisfaction with the effectiveness and inconvenience of current treatment modalities.

A common treatment modality for patients with psoriasis or vitiligo is to receive phototherapy administered in phototherapy centers. Patients are exposed to narrowband (NB) or broadband (BB) ultraviolet light B (UVB) or a therapy of psoralen plus ultraviolet light A (PUVA). Psoriasis patients require three or more sessions per week for approximately 30 treatments, and vitiligo patients require even more treatments. The use of phototherapy can be limited by travel distance from a phototherapy center and poor compliance to the necessary regimen of regular treatment sessions. Adjunctive therapies (e.g., topical prescriptions, over-the-counter topical creams) may improve the efficiency of phototherapy, but do not remove the necessity for phototherapy itself.

Ultraviolet light improves the symptoms of psoriasis through immunomodulatory mechanisms. The treatment of atopic dermatitis and alopecia greata with UV light has also been studied but not to the same degree; however, similar immunomodulatory mechanisms are likely. Leukoderma and vitiligo rely on the UV light to help re-pigment the skin because of a lack of melanin/melanocytes.

### SUMMARY

In one embodiment, a phototherapy apparatus is described for applying targeted phototherapy to a treatment area on human skin. The apparatus includes a UVB module arranged to output UVB light at about 308 nm. The UVB module has at least one flash lamp arranged to generate light including UVB light and a UVB transmissive window for transmitting UVB light generated by the at least one flash lamp. A user interface, which may include but is not limited to a pushbutton, is provided. A controller is configured to detect when the user interface is actuated and to cause pulsing of the at least one flash lamp in response to the actuation of the user interface.

In one variation, the phototherapy apparatus may include at least one sensor, and the controller may be configured to regulate one or more of the intensity, pulse rate, and pulse duration of the at least one flash lamp based on a signal from the at least one sensor. The sensor can include one or more of a melanin level sensor, an erythema sensor, a radiometer, and an infrared thermometer.

The at least one flash lamp may be configured to generate narrow-band UVB light that is transmissible through the window. Alternatively, the UVB module may further include a UVB band-pass filter adjacent to or incorporated into the window, and the at least one flash lamp may be configured to generate broad-band light including UVB light that is transmissible through the window and band-pass filter. The UVB module may include a reflective shield configured to redirect the UVB light generated by the at least one flash lamp through the window.

A contact sensor may be provided, and the controller configured to detect whether the contact sensor indicates that the apparatus is in contact with the skin of a user and to disable pulsing of the at least one flash lamp when the apparatus is not in contact with the skin of a user.

A cooling device may be provided that can be removably mounted to the UVB module to provide cooling to the skin surrounding the treatment area. Additionally, a removably mounted aperture may be provided to focus the UVB light to a treatment area having a desired shape and size.

In another embodiment, a method of applying targeted phototherapy to a treatment area on human skin includes contacting a phototherapy apparatus with the human skin, the phototherapy apparatus including at least one flash lamp and a UVB transmissive window, such that the window is positioned in alignment with the treatment area. The method further includes activating the at least one flash lamp to cause pulsed UVB light at 308 nm to be transmitted through the window.

In one variation, the method further includes sensing at least one parameter and regulating one or more of the intensity, pulse rate, and pulse duration of the at least one flash lamp based on the sensed at least one parameter. The parameter may be one or more of melanin level, erythema, radiation emitted by the flash lamp, and temperature of the treatment area.

The method may further include detecting whether the apparatus is in contact with the skin of a user and disabling pulsing of the at least one flash lamp when the apparatus is not in contact with the skin of a user. Additionally, the skin surrounding the treatment area may be cooled during pulsing of the at least one flash lamp.

In another embodiment, a phototherapy apparatus is described for applying targeted phototherapy to a treatment area on human skin. The apparatus includes a UVB module arranged to output UVB light at about 308 nm. The UVB module has at least one flash lamp arranged to generate light including UVB light, a UVB transmissive window for transmitting UVB light generated by the at least one flash lamp, at least one sensor selected from the group consisting of a melanin level sensor, an erythema sensor, a radiometer, and an infrared thermometer, and a contact sensor. The apparatus further includes a user interface and a controller configured to detect when the user interface is actuated and the contact sensor indicates that the apparatus is in contact with the skin of a user, to cause pulsing of the at least one flash lamp in response to the actuation of the user interface, and disable pulsing of the at least one flash lamp when the apparatus is not in contact with the skin of a user.

### BRIEF DESCRIPTION OF THE DRAWINGS

The foregoing, as well as the following detailed description of the embodiments, will be better understood when read in conjunction with the appended drawings. For the purpose of illustration, there are shown in the drawings some embodiments which may be preferable. It should be understood, however, that the embodiments depicted are not limited to the precise arrangements and instrumentalities shown.

FIG. 1 is a front/side perspective view showing an embodiment of a hand-held UV phototherapy apparatus.

FIG. 2 is a front/side exploded perspective view of an embodiment of a hand-held UV phototherapy apparatus.

FIG. 3 is a side/front perspective view of an embodiment of a hand-held UV phototherapy apparatus with a removable cooling device positioned around the periphery of the treatment area.

FIG. 4 is a front perspective view of an embodiment of an removable aperture for mounting on a hand-held UV phototherapy apparatus.

FIG. 5 is a block diagram depicting control components of an embodiment of a hand-held UV phototherapy device.

### DETAILED DESCRIPTION

A hand-held UVB phototherapy device is disclosed herein which can be used for home administered maintenance phototherapy. Home administered therapy can be use to treat various dermatoses, including psoriasis, vitiligo, leukoderma, atopic dermatitis, and alopecia greata. Home administered therapy may help to maintain remission once professional in-center treatment regimen has ended, or may be used to increase the time between professional in-center treatments. A hand-held UVB therapy apparatus provides targeted phototherapy, which spares healthy tissue from long-term cumulative UV exposure.

An embodiment of a UVB phototherapy apparatus 10 is depicted in FIG. 1. In the depicted embodiment, the apparatus 10 includes a housing 12 containing electronic components (not shown) for powering and controlling the apparatus 10, a power cord 11 for supplying power to the electronic components, and a removable UVB module 20 that includes one or more flash lamps 22 for generating broad-band or narrow-band UVB light.

A front cover 16 on the UVB module includes a bezel 17 surrounding a window 18 for transmitting UVB light generated by the one or more flash lamps 22 located within the UVB module 20 behind the window 18. The front cover 16 defines a contact surface of the apparatus 10 for contacting the skin of a patient to be treated. The window 18 is made from a UVB transmissive material and may serve as a UVB band-pass filter to block the transmission of other non-therapeutic wavelengths of light. In particular, the window 18 may include a band-pass filter that allows transmission of only light at about 308 nm. The window 18 enables targeted application of UVB phototherapy to treat dermatoses while minimizing the exposure of healthy skin.

A user interface 14, which may be in the form of a pushbutton, is mounted on the housing 12 for operating the apparatus 10. The user interface 14 can include a momentary contact switch for delivering pulses of light on demand, or for activating a control system that delivers a rapid series of pulses as long as the user interface 14 is actuated.

An exploded view of the apparatus 10 is shown in FIG. 2. The removable front cover 16 is removed to show the UVB module 20 mounted in the housing 12. The UVB module 20 is positioned behind the window 18 and bezel 17 when the front cover 16 is installed on the housing 12.

The UVB module 20 includes the one or more flash lamps 22. In the depicted embodiment, two flash lamps 22 are provided. The flash lamps 22 are preferably generally cylindrical or tubular lamps of the type that emit light along their length in all directions. The flash lamps 22 emit ultraviolet light that is primarily concentrated in the UVB range of 280 nm to 315 nm, is preferably concentrated within the anti-psoriatic region of 296 nm to 313 nm, and is more preferably concentrated at about 308 nm.

In one embodiment, the flash lamps 22 are gas-filled lamps that emit a spectrum of ultraviolet light. The gas is preferably a mixture containing a noble gas (e.g., argon, krypton, or xenon) and a halogen (e.g., fluorine or chlorine). In one embodiment, helium and/or neon is used as buffer gas, a halogen gas including primarily xenon chloride (XeCI) is used to produce pulsed light specifically at 308 nm.

In another embodiment, the flash lamps 22 are filament lamps that emit a broadband spectrum of light. Preferably, the filament is tungsten and the gas is a halogen gas (e.g., fluorine or chlorine). A band-pass filter in or adjacent to the window 18 may be used in conjunction with a broadband filament lamp to limit the light emitted from the device to the UVB range specifically to about 308 nm.

The UVB module 20 further includes a UVB light reflective shield 24. The one or more flash lamps 22 are positioned in front of the reflective shield 24 so that the light emitted by the lamps 22 is directed forward, toward (and through) the window 18 and/or band-pass filter. The reflective shield 24 includes several ports 30 in which various sensors can be mounted, including but not limited to a melanin level sensor 32, an erythema sensor 34, a radiometer 36, and an contactless infrared thermometer 38. The ports 30 are positioned so that, if necessary, any sensors mounted in the ports 30 have a clear optical path through the window 18. In addition, a spring-actuated contact sensor 40 is provided behind the front cover 16 to sense when the front cover 16 is placed into contact with the skin of a patient.

As shown in FIG. 5, a controller 50 receives input signals from the melanin level sensor 32, the erythema sensor 34, the radiometer 36, the infrared thermometer 38, the spring-actuated contact sensor 40, and the user interface 14, and controls pulsation of the flash lamps 22 via a capacitive pulse unit 52. The controller 50 and the capacitive pulse unit 52 are powered by a power supply 54. As shown, the power supply 54 may be powered via an AC power cord 11, or by a replaceable and/or rechargeable battery so as to make the apparatus 10 more readily portable.

The controller 50 may regulate or limit the irradiance (intensity/output levels) of the flash lamps 22 based on input signals from one or more of the melanin level sensor 32 and the erythema sensor 34. The controller may limit the intensity and duration of radiation exposure based on input signals from the infrared thermometer 38, if skin temperature in the treatment region gets too high. The controller 50 may also include a timer or energy counter so that the dose or fluence emitted by the apparatus 10 can be set in advance, or limited during use. Alternatively, the controller 50 can monitor the lamp output through the radiometer 36 to ensure proper dosing.

The controller 50 can further be configured to set a flash lamp intensity, a pulse on-time, and a number of pulses per exposure so that both the irradiance (intensity) and fluence (dose) can be optimized for the particular patient and condition being treated.

Based on input from the contact sensor 40, the controller 50 limits or prevents firing of the flash lamps 22 unless the front cover 16 is in contact with the skin with sufficient force (minimal) that the contact sensor 40 is actuated.

FIG. 3 shows the phototherapy apparatus 10 with a cooling device 60 removably mounted over the front cover 16 to provide cooling to the skin around the periphery of the treatment area. The cooling device 60 includes cooling elements 62 adapted to be in contact with the skin and to deliver cooling to the skin, as well as a heat dissipation structure 64 to remove heat from the cooling elements 62. The cooling elements 62 may be thermoelectric chillers, or may be chilled by another mechanism, including by the flow of liquid or cryogenic coolant. Cooling helps to reduce the skin temperature at and around the treatment area, and helps to control pain so that a full course of treatment can be applied.

FIG. 4 shows an aperture 70 that can be removably attached to the front cover 16 of the phototherapy apparatus 10 to assist in focusing the UVB radiation on the desired treatment area. The aperture 70, as shown, includes a base 72, a rim 74, a tapered portion 76, and an opening 80 that is smaller than the window 18 in the front cover 16. The rim 74 may be used to enable the aperture 70 to attach to or snap onto the front cover 16, and may engage the bezel 17 or another portion of the front cover 16. The opening 80 may be of any shape or size. The opening 80 may be completely open, or may include a UVB transparent window or band-pass filter. The aperture 70 may include a reflective inner surface to improve the energy transmission through the opening 80.

In operation, the phototherapy apparatus 10 is positioned so that the window 18, or the opening 80 in the aperture 70 if appropriate, is aligned with an area of the skin to be treated. The front cover 16 (or aperture 70) is placed into contact with the skin so that the contact sensor 40 is actuated. The user interface 14 is actuated, and if required held in an actuated state, to activate the controller 50. The controller 50 sends power to the capacitive pulse unit 52, which begins pulsing the flash lamps 22 at an intensity, rate, and duration as commanded by the controller 50. Should the phototherapy apparatus 10 be moved so that the contact sensor 40 is no longer actuated, pulsing of the flash lamps 22 ceases.

In the case where treatment of fixed duration is to be applied, the user interface 14 need not be held in an actuated state for pulsation of the flash lamps 22 to continue for the fixed duration. In the case where treatment is of variable duration, or manually controlled, release of the pushbutton will cease flashing of the lamps 22.

The controller 50 may increase or decrease the intensity, rate, or duration of light pulses based on feedback from the melanin level sensor 32, the erythema sensor 32, the radiaometer 34, and/or the infrared thermometer 36, as needed, to optimize the effectiveness and minimize the length of the treatment, and to ensure patient safety.

While reference has been made to specific embodiments, it is apparent that other embodiments and variations can be devised by others skilled in the art without departing from their spirit and scope. The appended claims are intended to be construed to include all such embodiments and equivalent variations.

What is claimed is:

1. A phototherapy apparatus for applying targeted phototherapy to a treatment area on human skin, comprising:
a UVB module arranged to output UVB light at about 308 nm, comprising:
at least one flash lamp arranged to generate light including UVB light; and
a UVB transmissive window for transmitting UVB light generated by the at least one flash lamp;
a user interface; and
a controller configured to detect when the user interface is actuated and to cause pulsing of the at least one flash lamp in response to the actuation of the user interface.

2. The phototherapy apparatus of claim 1,
the UVB module further comprising at least one sensor;
wherein the controller is configured to regulate one or more of the intensity, pulse rate, and pulse duration of the at least one flash lamp based on a signal from the at least one sensor.

3. The phototherapy apparatus of claim 2, wherein the at least one sensor includes a sensor selected from the group consisting of: a melanin level sensor, an erythema sensor, a radiometer, and an infrared thermometer.

4. The phototherapy apparatus of claim 1,
wherein the at least one flash lamp is configured to generate narrow-band UVB light that is transmissible through the window.

5. The phototherapy apparatus of claim 1,
the UVB module further comprising a UVB band-pass filter adjacent to or incorporated into the window;
wherein the at least one flash lamp is configured to generate broad-band light including UVB light that is transmissible through the window and band-pass filter.

6. The phototherapy apparatus of claim 1,
the UVB module further comprising a reflective shield configured to redirect the UVB light generated by the at least one flash lamp through the window.

7. The phototherapy apparatus of claim 1,
the UVB module further comprising a contact sensor;
wherein the controller is configured to detect whether the contact sensor indicates that the apparatus is in contact with the skin of a patient and to disable pulsing of the at least one flash lamp when the apparatus is not in contact with the skin of a patient.

8. The phototherapy apparatus of claim 1, further comprising a cooling device configured to be removably mounted to the UVB module to provide cooling to the skin surrounding the treatment area.

9. The phototherapy apparatus of claim 1, further comprising an aperture configured to be removably mounted to the UVB module and to focus the UVB light to a treatment area having a desired shape and size.

10. A method of applying targeted phototherapy to a treatment area on human skin, comprising:
contacting a phototherapy apparatus with the human skin, the phototherapy apparatus including at least one flash lamp and a UVB transmissive window, such that the window is positioned in alignment with the treatment area; and
activating the at least one flash lamp to cause pulsed UVB light at 308 nm to be transmitted through the window.

11. The method of claim 10, further comprising:
sensing at least one parameter; and
regulating one or more of the intensity, pulse rate, and pulse duration of the at least one flash lamp based on the at least one sensed parameter.

12. The method of claim 11, wherein the at least one parameter is selected from the group consisting of: melanin level, erythema, radiation emitted by the flash lamp, and temperature of the treatment area.

13. The method of claim 10,
wherein the at least one flash lamp is configured to generate narrow-band UVB light that is transmissible through the window.

14. The method of claim 10, wherein the at least one flash lamp is configured to generate broad-band UVB light, further comprising:
filtering the broad-band UVB light through a band-pass filter.

15. The method of claim 10, further comprising:
detecting whether the apparatus is in contact with the skin of a patient; and
disabling pulsing of the at least one flash lamp when the apparatus is not in contact with the skin of a patient.

16. The method of claim 10, further comprising cooling to the skin surrounding the treatment area during pulsing of the at least one flash lamp.

17. The method of claim 10, further comprising focusing the UVB light to a treatment area having a desired shape and size.

18. A phototherapy apparatus for applying targeted phototherapy to a treatment area on human skin, comprising:
a UVB module arranged to output UVB light at about 308 nm, comprising:
at least one flash lamp arranged to generate light including UVB light;
a UVB transmissive window for transmitting UVB light generated by the at least one flash lamp;
at least one sensor selected from the group consisting of: a melanin level sensor, an erythema sensor, a radiometer, and an infrared thermometer; and
a contact sensor;
a user interface; and
a controller configured to:
detect when the user interface is actuated and the contact sensor indicates that the apparatus is in contact with the skin of a patient;
cause pulsing of the at least one flash lamp in response to the actuation of the user interface; and
disable pulsing of the at least one flash lamp when the apparatus is not in contact with the skin of a patient.
